(19) United States
(12) Patent Application Publication
(10) Pub. No.: US 2023/0181630 A1
(43) Pub. Date: Jun. 15, 2023

(54) PACKAGED COMPOSITIONS COMPRISING HYPOCHLOROUS ACID AND MENTHOL AND METHODS OF MAKING THEREOF

(71) Applicant: Harrow IP, LLC, Nashville, TN (US)

(72) Inventors: Dennis Saadeh, Nashville, TN (US); Mark Baum, Nashville, TN (US)

(21) Appl. No.: 18/078,350

(22) Filed: Dec. 9, 2022

Related U.S. Application Data
(60) Provisional application No. 63/288,358, filed on Dec. 10, 2021.

(51) Int. Cl. A61K 33/20; A61K 36/534; A61K 31/05; A61K 31/164; A61K 9/00; A61K 9/70

(52) U.S. Cl. CPC A61K 33/20; A61K 36/534; A61K 31/05; A61K 31/164; A61K 9/0048; A61K 9/7015

(57) ABSTRACT

The present disclosure relates to packaged eyelid scrubs as well as methods for making the packaged eyelid scrubs. The packaged eyelid scrubs include a pharmaceutical composition that includes hypochlorous acid and menthol, and a package that includes an opaque airless spray container filled with the pharmaceutical composition. Methods of cleansing an eyelid using the pharmaceutical compositions are also described herein.

### CROSS-REFERENCE TO RELATED APPLICATIONS

This application claims priority to U.S. Provisional Application No. 63/288,358 entitled “PACKAGED COMPOSITIONS COMPRISING HYPOCHLOROUS ACID AND MENTHOL AND METHODS OF MAKING THEREOF” filed Dec. 10, 2021, the entire contents of which are incorporated by reference herein.

### FIELD OF THE DISCLOSURE

The present disclosure relates to compositions comprising hypochlorous acid and menthol for use in eyelid scrubs. Therefore, the present disclosure relates to the fields of medicine, pharmacy, and ophthalmology.

### BACKGROUND

Dry eyes and inflammation caused by numerous diseases and disorders affect many patients. Aside from treating the underlying condition, treatments can include applying an eyelid scrub to the irritated eye. Generally, eyelid scrubs include cleansing agents that clean the area and may provide relief over time, but generally fail to provide short term relief from pain or discomfort.

Hypochlorous acid is one exemplary cleansing agent that has been used in skin cleansing treatments. However, it has been shown that hypochlorous acid is unstable when exposed to ultraviolet light, air, and/or elevated temperature. Thus, over time, hypochlorous acid products become less effective as the hypochlorous acid degrades.

There is a need, therefore, for a shelf-stable eyelid scrub that cleanses an irritated eyelid while also providing relief from pain or discomfort.

### SUMMARY OF THE DISCLOSURE

Described herein is a packaged eyelid or eyelash scrub composition that includes a pharmaceutical composition comprising hypochlorous acid and menthol, wherein the pharmaceutical composition has a water-like viscosity, and a package comprising an opaque airless spray container filled with the pharmaceutical composition, such that the pharmaceutical composition is stable for greater than 30 days after the airless spray container is actuated for the first time.

In some embodiments, the menthol may be L-menthol. In some embodiments, the L-menthol may have a concentration in the pharmaceutical composition from about 0.01% (w/w) to about 0.03% (w/w). In some examples, the L-menthol may have a concentration in the pharmaceutical composition of about 0.025% (w/w).

In some embodiments, the hypochlorous acid may have a concentration from about 0.01% (w/w) to about 0.05% (w/w). In some examples, the hypochlorous acid may have a concentration of about 0.02% (w/w).

In some embodiments, the pharmaceutical composition further includes at least one surfactant. In some additional embodiments, the at least one surfactant forms micelles. In some aspects, the at least one surfactant is a non-ionic surfactant, an anionic surfactant, or combinations thereof. In some additional aspects, the anionic surfactant may be sodium lauryl sarcosinate. In still additional aspects, the non-ionic surfactant may be sorbitan monolaurate. In still further aspects, the at least one surfactant may have a concentration from about 0.1% (w/w) to about 2% (w/w). In some examples, the surfactant may have a concentration in the pharmaceutical composition of about 1% (w/w).

In some embodiments, the composition further includes a pH modifier. In some aspects, the pH modifier may have selected from the group consisting of sodium hydroxide and hydrochloric acid. In some embodiments, the pH of the pharmaceutical composition may be from about 4 and about 7.

In some embodiments, the opaque airless spray container may include glass, polyethylene terephthalate, low density polyethylene, polypropylene, or combinations thereof. In some additional embodiments, the opaque airless spray container may meet USP acceptance criteria for light-resistant packaging components. In still further embodiments, the percentage of spectral transmission of the opaque airless spray container at any wavelength of light between 290 nm and 450 nm is 25% or less. In some aspects, the spectral transmission of the opaque airless spray container at any wavelength of light between 290 nm and 450 nm is 10% or less.

In some embodiments, the hypochlorous acid has a concentration in the pharmaceutical composition of at least about 200 ppm for at least 30 days after the opaque airless spray container is actuated for the first time. In some preferred embodiments, the hypochlorous acid has a concentration in the pharmaceutical composition of at least about 200 ppm for at least 60 days. In more preferred embodiments, the hypochlorous acid has a concentration in the pharmaceutical composition of at least about 200 ppm for at least 90 days.

In some embodiments, the pharmaceutical composition may further include an active ingredient. In some aspects, the active ingredient may be selected from the group consisting of an antibiotic, an antiparasitic, a steroid, and combinations thereof.

In some embodiments, the antibiotic may be selected from the group consisting of a tetracycline, a fluoroquinolone, a macrolide, or combinations thereof. In some aspects, the tetracycline may be selected form the group consisting of doxycycline, minocycline, and combinations thereof.

In some embodiments, the antiparasitic may be selected from the group consisting of ivermectin, permethrin, and combinations thereof.

In some embodiments, the steroid may be selected from the group consisting of hydrocortisone, dexamethasone, triamcinolone, prednisolone, and combinations thereof.

In some embodiments, the pharmaceutical composition may further include sterile water.

In some embodiments, the pharmaceutical composition may further comprise polyethylene glycol. In some aspects, the polyethylene glycol may have a molar mass of about 400.

In some embodiments, the water-like viscosity of the pharmaceutical composition may be from about 1 cP and about 10 cP. In some preferred embodiments, the water-like viscosity of the pharmaceutical composition may be from about 1 cP and about 5 cP.

Further described herein is a method for cleansing and soothing an irritated eyelid. The method includes providing a packaged eyelid scrub composition of the present disclosure, actuating the opaque airless spray container, and applying the pharmaceutical composition to the irritated eyelid. In some embodiments, the method may be efficacious in treating dry eye, meibomian gland dysfunction, blepharitis, demodicosis, and/or eczema. In some embodiments, the step of applying the pharmaceutical composition to the irritated eyelid may be performed at least once per day. In some aspects, the step of applying the pharmaceutical composition to the irritated eyelid may be performed at least twice, at least three times, or at least four times per day.

In some embodiments, the step of actuating the opaque airless spray container may release about 0.1 mL to about 0.2 mL of the pharmaceutical composition.

In some embodiments, the step of actuating the opaque airless spray container may be performed one or more times before the step of applying the pharmaceutical composition to the irritated eyelid.

In some embodiments, the method may further include providing an absorbing article. In some aspects, the absorbing article may include a cotton ball, a cotton oval, or a cotton swab.

In some embodiments, the step of actuating the opaque airless spray container may be performed in a manner such that the pharmaceutical composition is released onto and absorbed by the absorbing article.

In some embodiments, the step of applying the pharmaceutical composition to the irritated eyelid may be accomplished by applying the absorbing article to the irritated eyelid.

Further described herein is a method of treating dry eye, meibomian gland dysfunction, blepharitis, demodicosis, and/or eczema in a patient's eyelid. The method includes providing a packaged eyelid scrub composition of the present disclosure, actuating the opaque airless spray container, and applying the pharmaceutical composition to the patient's eyelid.

Further described herein is a method of removing makeup. The method includes providing a packaged eyelid scrub composition of the present disclosure, providing an absorbing article, actuating the opaque airless spray container such that the pharmaceutical composition is released onto the cloth, and rubbing the cloth on makeup-covered skin.

Further described herein is a method of making the packaged eyelid scrub composition of the present disclosure. The method includes providing a first solution comprising menthol and a non-aqueous solvent, providing a second solution comprising hypochlorous acid, adding a surfactant to the second solution, mixing the second solution until the second solution is homogenous, adding the first solution to the second solution, resulting in a third solution, mixing the third solution is homogenous, adding a pH adjusting agent to adjust the pH of the third solution until the pH of the third solution is from about 4 to about 7, adding sterile water to the third solution, and packaging the third solution in an opaque airless spray container.

### DETAILED DESCRIPTION

Before the present invention is disclosed and described, it is to be understood that this invention is not limited to the particular methods, compositions, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Concentrations, amounts, and other numerical data may be expressed or presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 2 to about 50” should be interpreted to include not only the explicitly recited values of 2 to 50, but also include all individual values and sub-ranges within the indicated range. Thus, included in this numerical range are individual values such as 2, 2.4, 3, 3.7, 4, 5.5, 10, 10.1, 14, 15, 15.98, 20, 20.13, 23, 25.06, 30, 35.1, 38.0, 40, 44, 44.6, 45, 48, and sub-ranges such as from 1-3, from 2-4, from 5-10, from 5-20, from 5-25, from 5-30, from 5-35, from 5-40, from 5-50, from 2-10, from 2-20, from 2-30, from 2-40, from 2-50, etc. This same principle applies to ranges reciting only one numerical value as a minimum or a maximum. Furthermore, such an interpretation should apply regardless of the breadth of the range or the characteristics being described.

As used herein, the term “about” is used to provide flexibility to a numerical range endpoint by providing that a given value may be “a little above” or “a little below” the endpoint. For example, the endpoint may be within 10%, 8%, 5%, 3%, 2%, or 1% of the listed value. Further, for the sake of convenience and brevity, a numerical range of “about 50 mg/mL to about 80 mg/m L” should also be understood to provide support for the range of “50 mg/mL to 80 mg/m L.” The endpoint may also be based on the variability allowed by an appropriate regulatory body, such as the FDA, USP, etc.

In this disclosure, “comprises,” “comprising,” “containing,” and “having” and the like can have the meaning ascribed to them in U.S. Patent Law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. In this specification when using an open ended term, like “comprising” or “including,” it is understood that direct support should be afforded also to “consisting essentially of” language as well as “consisting of” language as if stated explicitly and vice versa.

As used herein, “stable” and “stability” refers to the slow or lacking degradation of one or more active ingredients of a pharmaceutical composition. Stated another way, a stable composition is one in which one or more active ingredients have not degraded or decomposed over a period of time and the concentration of one or more active ingredients has not significantly changed over a period of time. Alternatively, or in addition, a stable composition has a pH that does not change significantly over time. For example, a stable composition's pH may not change by more than ±0.5 during the shelf-life of the composition.

As used herein, “potent” and “potency” refers to the concentration of one or more active ingredients in a pharmaceutical composition in reference to the amount of the one or more active ingredients needed to produce a therapeutic effect. As a non-limiting example, a hypothetical pharmaceutical composition may require 1% (w/v) of an active ingredient to have a therapeutic effect. The hypothetical composition would lack potency if it only contained 0.1% (w/v) of the active ingredient. Generally, a stable composition is also a potent composition.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

Described herein are packaged compositions comprising hypochlorous acid and menthol and methods of making thereof. Generally, the packaged composition includes a pharmaceutical composition and a package. The pharmaceutical composition includes hypochlorous acid and menthol. The pharmaceutical composition also has a water-like viscosity. The package is an opaque airless spray container that is filled with the pharmaceutical composition. In a preferred embodiment of the present disclosure, the pharmaceutical composition may be stable for greater than 30 days after the airless spray container is actuated for the first time.

Further described herein are methods of making the packaged composition of the present disclosure.

Further described herein are methods of using the packaged composition. The packaged composition may be used to cleanse and sooth an irritated eyelid. The packaged composition may further be used to remove makeup.

### I. Packaged Composition

Described herein are packaged compositions comprising hypochlorous acid and menthol. The packaged composition includes a pharmaceutical composition and a package.

A. Pharmaceutical Composition

The packaged composition includes a pharmaceutical composition. The pharmaceutical composition of the present disclosure includes hypochlorous acid and menthol.

The pharmaceutical composition also has a water-like viscosity. In some embodiments, the pharmaceutical composition may have a viscosity from about 1 cP to about 10 cP. In some aspects, the pharmaceutical composition may have a viscosity from about 1 cP to about 7 cP, about 1 cP to about 5 cP, or about 1 cP to about 3 cP. In some additional aspects, the pharmaceutical composition may have a viscosity from about 1 cP to about 1.5 cP, about 1.5 cP to about 2 cP, about 2 cP to about 2.5 cP, about 2.5 cP to about 3 cP, about 3 cP to about 3.5 cP, about 3.5 cP to about 4 cP, about 4 cP to about 4.5 cP, about 4.5 cP to about 5 cP, about 5 cP to about 5.5 cP, about 5.5 cP to about 6 cP, about 6 cP to about 6.5 cP, about 6.5 cP to about 7 cP, about 7 cP to about 7.5 cP, about 7.5 cP to about 8 cP, about 8 cP to about 8.5 cP, about 8.5 cP to about 9 cP, about 9 cP to about 9.5 cP, or about 9.5 cP to about 10 cP. In still additional aspects, the pharmaceutical composition may have a viscosity of about 1 cP, 1.5 cP, 2 cP, 2.5 cP, 3 cP, 3.5 cP, 4 cP, 4.5 cP, 5 cP, 5.5 cP, 6 cP, 6.5 cP, 7 cP, 7.5 cP, 8 cP, 8.5 cP, 9 cP, 9.5 cP, or about 10 cP. In some examples, the pharmaceutical composition may have a viscosity of greater than 10 cP, such as about 20 cP, about 30 cP, about 40 cP, or about 50 cP.

i. Hypochlorous Acid

The pharmaceutical composition of the present disclosure includes hypochlorous acid. Hypochlorous acid (HOCl) is a weak acid that is formed when chlorine dissolves in water. Hypochlorous acid is also a known antiseptic effective against many kinds of microorganisms, including bacteria, viruses, fungi, and parasites.

The hypochlorous acid may be formed by the addition of a hypochlorite salt to an aqueous solution and acidifying the solution to a pH of 6 or below. The hypochlorite salt may be sodium hypochlorite, calcium hypochlorite, or other hypochlorite salts known in the art. The hypochlorous acid may also be formed by electrolyzing sodium chloride. Other methods of making and procuring hypochlorous acid are well known in the art.

In some embodiments, the hypochlorous acid may have a concentration in the pharmaceutical composition from about 0.01% (w/w) to about 0.05% (w/w). In some aspects, the hypochlorous acid may have a concentration in the pharmaceutical composition from about 0.01% (w/w) to about 0.04% (w/w), about 0.01% (w/w) to about 0.03% (w/w), or about 0.015% (w/w) to about 0.025% (w/w). In some additional aspects, the hypochlorous acid may have a concentration in the pharmaceutical composition from about 0.01% (w/w) to about 0.015% (w/w), about 0.015% (w/w) to about 0.02% (w/w), about 0.02% (w/w) to about 0.025% (w/w), about 0.025% (w/w) to about 0.03% (w/w), about 0.03% (w/w) to about 0.035% (w/w), about 0.035% (w/w) to about 0.04% (w/w), about 0.04% (w/w) to about 0.045% (w/w), or about 0.045% (w/w) to about 0.05% (w/w). In yet additional aspects, the hypochlorous acid may have a concentration of about 0.01% (w/w), 0.015% (w/w), 0.02% (w/w), 0.025% (w/w), 0.03% (w/w), 0.035% (w/w), 0.04% (w/w), 0.045% (w/w), or about 0.05% (w/w). In some examples, the hypochlorous acid may have a concentration in the pharmaceutical composition of about 0.02% (w/w).

ii. Menthol

The pharmaceutical composition of the present disclosure includes menthol. Menthol provides a soothing and/or cooling effect when applied to the skin, which may provide pain relief.

The menthol may include any of the known stereoisomers of menthol, including L-menthol, L-isomenthol, L-neomenthol, L-neoisomenthol, D-menthol, D-isomenthol, D-neomenthol, or D-neoisomenthol, or combinations thereof. In some examples, the pharmaceutical composition of the present disclosure includes L-menthol.

In some embodiments, the menthol may have a concentration in the pharmaceutical composition from about 0.01% (w/w) to about 0.03% (w/w). In some aspects, the menthol may have a concentration in the pharmaceutical composition from about 0.015% (w/w) to about 0.03% (w/w), about 0.02% (w/w) to about 0.03% (w/w), or from about 0.0225% (w/w) to about 0.0275% (w/w). In some additional aspects, the menthol may have a concentration in the pharmaceutical composition from about 0.01% (w/w) to about 0.0125% (w/w), about 0.0125% (w/w) to about 0.015% (w/w), about 0.015% (w/w) to about 0.0175% (w/w), about 0.0175% (w/w) to about 0.02% (w/w), about 0.02% (w/w) to about 0.0225% (w/w), about 0.0225% (w/w) to about 0.025% (w/w), about 0.025% (w/w) to about 0.0275% (w/w), or about 0.0275% (w/w) to about 0.03% (w/w). In still additional aspects, the menthol may have a concentration in the pharmaceutical composition from about 0.01% (w/w), 0.011% (w/w), 0.012% (w/w), 0.013% (w/w), 0.014% (w/w), 0.015% (w/w), 0.016% (w/w), 0.017% (w/w), 0.018% (w/w), 0.019% (w/w), 0.02% (w/w), 0.021% (w/w), 0.022% (w/w), 0.023% (w/w), 0.024% (w/w), 0.025% (w/w), 0.026% (w/w), 0.027% (w/w), 0.028% (w/w), 0.029% (w/w), or about 0.03% (w/w). In some examples, the menthol is L-menthol having a concentration of about 0.025% (w/w).

iii. Surfactant

In some embodiments, the pharmaceutical composition of the present disclosure may include at least one surfactant. The at least one surfactant may be a non-ionic surfactant, an anionic surfactant, or combinations thereof. In some embodiments, the at least one surfactant forms micelles in the pharmaceutical composition.

The at least one surfactant may have a concentration in the pharmaceutical composition from about 0.1% (w/w) to about 2% (w/w). In some aspects, the at least one surfactant may have a concentration in the pharmaceutical composition from about 0.25% (w/w) to about 1.75% (w/w), 0.5% (w/w) to about 1.5% (w/w), or about 0.75% (w/w) to about 1.25% (w/w). In some additional aspects, the at least one surfactant may have a concentration in the pharmaceutical composition from about 0.1% (w/w) to about 0.25% (w/w), about 0.25% (w/w) to about 0.5% (w/w), about 0.5% (w/w) to about 0.75% (w/w), about 0.75% (w/w) to about 1% (w/w), about 1% (w/w) to about 1.25% (w/w), about 1.25% (w/w) to about 1.5% (w/w), about 1.5% (w/w) to about 1.75% (w/w), or about 1.75% (w/w) to about 2% (w/w). In still additional aspects, the at least one surfactant may have a concentration in the pharmaceutical composition of about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), or about 2% (w/w). In some examples, the at least one surfactant has a concentration in the pharmaceutical composition of about 1% (w/w).

In some embodiments, the at least one surfactant may be a non-ionic surfactant. The non-ionic surfactant may include sorbitan monooleate, sorbitan monolaurate, sorbitan monostearate, sorbitan tristearate, polysorbates, ethoxylates, poloxamers, fatty acid esters of glycerol, fatty acid esters of sorbitol, Tweens, or other non-ionic surfactants known in the art. In some examples, the non-ionic surfactant is sorbitan monooleate. In some additional examples, the non-ionic surfactant is sorbitan monolaurate.

In some embodiments, the non-ionic surfactant may have a concentration in the pharmaceutical composition from about 0.1% (w/w) to about 2% (w/w). In some aspects, the non-ionic surfactant may have a concentration in the pharmaceutical composition from about 0.25% (w/w) to about 1.75% (w/w), 0.5% (w/w) to about 1.5% (w/w), or about 0.75% (w/w) to about 1.25% (w/w). In some additional aspects, the non-ionic surfactant may have a concentration in the pharmaceutical composition from about 0.1% (w/w) to about 0.25% (w/w), about 0.25% (w/w) to about 0.5% (w/w), about 0.5% (w/w) to about 0.75% (w/w), about 0.75% (w/w) to about 1% (w/w), about 1% (w/w) to about 1.25% (w/w), about 1.25% (w/w) to about 1.5% (w/w), about 1.5% (w/w) to about 1.75% (w/w), or about 1.75% (w/w) to about 2% (w/w). In still additional aspects, the non-ionic surfactant may have a concentration in the pharmaceutical composition of about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), or about 2% (w/w). In some examples, the non-ionic surfactant has a concentration in the pharmaceutical composition of about 1% (w/w).

In some embodiments, the at least one surfactant may be an anionic surfactant. The anionic surfactant may include sodium lauryl sarcosinate, ammonium lauryl sulfate, sodium lauryl sulfate, sodium myreth sulfate, sodium pareth sulfate, sodium stearate, α-olefin sulfonate, ammonium laureth sulfate, and other anionic surfactants known in the art. In some examples, the anionic surfactant is sodium lauryl sarcosinate.

In some embodiments, the anionic surfactant may have a concentration in the pharmaceutical composition from about 0.1% (w/w) to about 2% (w/w). In some aspects, the anionic surfactant may have a concentration in the pharmaceutical composition from about 0.25% (w/w) to about 1.75% (w/w), 0.5% (w/w) to about 1.5% (w/w), or about 0.75% (w/w) to about 1.25% (w/w). In some additional aspects, the anionic surfactant may have a concentration in the pharmaceutical composition from about 0.1% (w/w) to about 0.25% (w/w), about 0.25% (w/w) to about 0.5% (w/w), about 0.5% (w/w) to about 0.75% (w/w), about 0.75% (w/w) to about 1% (w/w), about 1% (w/w) to about 1.25% (w/w), about 1.25% (w/w) to about 1.5% (w/w), about 1.5% (w/w) to about 1.75% (w/w), or about 1.75% (w/w) to about 2% (w/w). In still additional aspects, the anionic surfactant may have a concentration in the pharmaceutical composition of about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), 1% (w/w), 1.1% (w/w), 1.2% (w/w), 1.3% (w/w), 1.4% (w/w), 1.5% (w/w), 1.6% (w/w), 1.7% (w/w), 1.8% (w/w), 1.9% (w/w), or about 2% (w/w). In some examples, the anionic surfactant has a concentration in the pharmaceutical composition of about 1% (w/w).

In some aspects, the at least one surfactant may be a combination of a non-ionic surfactant and an anionic surfactant. In some examples, the at least one surfactant includes sorbitan monolaurate and sodium lauryl sarcosinate. In some additional examples, the sorbitan monolaurate and sodium lauryl sarcosinate are present in the pharmaceutical in equal amounts based on mass.

iv. pH Modifier

In some embodiments, the pharmaceutical composition of the present disclosure may include a pH modifier. The pH modifier may be added to the pharmaceutical composition as necessary to bring the pharmaceutical composition to a desired pH level. In some aspects, the pH modifier may include hydrochloric acid or sodium hydroxide; however, other acids and bases may be used as will be appreciated by those having ordinary skill in the art.

The pharmaceutical composition may have a pH from about 4 to about 7. In some aspects, the pH of the pharmaceutical composition may have a pH from about 4 to about 4.5, about 4.5 to about 5, 5 to about 5.5, about 5.5 to about 6, about 6 to about 6.5, or about 6.5 to about 7. In some examples, the pharmaceutical composition may have a pH from about 4.5 and about 5.5.

v. Active Ingredient

In some embodiments, the pharmaceutical composition may include an active ingredient. The active ingredient may be an antibiotic, an antiparasitic, a steroid, or combinations thereof.

In some embodiments, the active ingredient is an antibiotic. The antibiotic may be a tetracycline, a fluoroquinolone, a macrolide, or combinations thereof.

In some aspects when the antibiotic is a tetracycline, the tetracycline may include doxycycline, minocycline, tetracycline, chlortetracycline, oxytetracycline, demeclocycline, lymecycline, meclocycline, methacycline, minocycline, rolitetracycline, tigecycline, eravacycline, sarecycline, omadacycline, or other tetracyclines known in the art and combinations thereof. In some examples, the tetracycline is doxycycline. In some additional examples, the tetracycline is minocycline.

The tetracycline may have a concentration in the pharmaceutical composition from about 0.5% (w/w) to about 3% (w/w). In some aspects, the tetracycline may have a concentration from about 0.5% (w/w) to 1% (w/w), about 1% (w/w) to about 1.5% (w/w), about 1.5% (w/w) to about 2% (w/w), or about 2.5% (w/w) to about 3% (w/w). In some additional aspects, the tetracycline may have a concentration in the pharmaceutical composition of about 0.5% (w/w), 1% (w/w), 1.5% (w/w), 2% (w/w), 2.5% (w/w), or about 3% (w/w). In some examples, the tetracycline may have a concentration of about 1% (w/w).

In some aspects when the antibiotic is a fluoroquinolone antibiotic, the fluoroquinolone may include besifloxacin, ciprofloxacin, gatifloxacin, levofloxacin, moxifloxacin, ofloxacin, or other fluoroquinolones known in the art and combinations thereof. In some examples, the fluoroquinolone is moxifloxacin. The fluoroquinolone may have a concentration in the pharmaceutical composition from about 0.3% (w/w) to about 1% (w/w). In some aspects, the fluoroquinolone may have a concentration in the pharmaceutical composition from about 0.3% (w/w) to about 0.4% (w/w), about 0.4% (w/w) to about 0.5% (w/w), about 0.5% (w/w) to about 0.6% (w/w), about 0.6% (w/w) to about 0.7% (w/w), about 0.7% (w/w) to about 0.8% (w/w), about 0.8% (w/w) to about 0.9% (w/w), or about 0.9% (w/w) to about 1% (w/w). In some additional aspects, the fluoroquinolone may have a concentration of about 0.3% (w/w), 0.4% (w/w), 0.5% (w/w), 0.6% (w/w), 0.7% (w/w), 0.8% (w/w), 0.9 (w/w), or about 1% (w/w). In some examples, the fluoroquinolone may have a concentration in the pharmaceutical composition of about 0.5% (w/w).

In some aspects when the antibiotic is a macrolide antibiotic, the macrolide antibiotic may include azithromycin, clarithromycin, fidaxomicin, erythromycin, or other macrolides known in the art and combinations thereof. The macrolide may have a concentration in the pharmaceutical composition from about 0.5% (w/w) to about 3% (w/w). In some aspects, the macrolide may have a concentration from about 0.5% (w/w) to 1% (w/w), about 1% (w/w) to about 1.5% (w/w), about 1.5% (w/w) to about 2% (w/w), or about 2.5% (w/w) to about 3% (w/w). In some additional aspects, the macrolide may have a concentration in the pharmaceutical composition of about 0.5% (w/w), 1% (w/w), 1.5% (w/w), 2% (w/w), 2.5% (w/w), or about 3% (w/w). In some examples, the macrolide may have a concentration of about 1% (w/w).

In some embodiments, the active ingredient is an antiparasitic. The antiparasitic may be an ectoparasiticide such as permethrin, ivermectin, crotamiton, benzyl benzoate, lindane, sulfur, dicophan, or other ectoparasiticides known in the art or combinations thereof. In particular, the ectoparasiticide may be effective to treat demodicosis. Demodicosis is usually caused by a sensitivity to and the overpopulation of demodex mites, particularly Demodex folliculorum. In some examples, the antiparasitic may include permethrin. In some additional examples, the antiparasitic may include ivermectin. The antiparasitic may have a concentration in the pharmaceutical composition from about 0.01% (w/w) to about 0.5% (w/w). In some aspects, the concentration of the antiparasitic may be from about 0.01% (w/w) to about 0.05% (w/w), about 0.05% (w/w) to about 0.1% (w/w), about 0.1% (w/w) to about 0.2% (w/w) about 0.2% (w/w) to about 0.3% (w/w), about 0.3% (w/w) to about 0.4% (w/w) or about 0.4% (w/w) to about 0.5% (w/w). In some additional aspects, the concentration of the antiparasitic in the pharmaceutical composition may be about 0.01% (w/w), 0.05% (w/w), 0.1% (w/w), 0.2% (w/w), 0.3% (w/w), 0.4% (w/w), or about 0.5% (w/w). In some examples, the antiparasitic may have a concentration of about 0.1% (w/w).

In some embodiments, the active ingredient is a steroid. The steroid may be a corticosteroid. In some aspects, the corticosteroid may include hydrocortisone, dexamethasone, triamcinolone, prednisolone, loteprednol etabonate, and combinations thereof. The steroid may have a concentration in the pharmaceutical composition from about 0.1% (w/w) to about 1% (w/w). In some aspects, the steroid may have a concentration in the pharmaceutical composition from about 0.1% (w/w) to about 0.2% (w/w), about 0.2% (w/w) to about 0.3% (w/w), about 0.3% (w/w) to about 0.4% (w/w), about 0.4% (w/w) to about 0.5% (w/w), about 0.5% (w/w) to about 0.6% (w/w), about 0.6% (w/w) to about 0.7% (w/w), about 0.7% (w/w) to about 0.8% (w/w), about 0.8% (w/w) to about 0.9% (w/w), or about 0.9% (w/w) to about 1% (w/w). In some additional aspects, the steroid may have a concentration of about 0.1% (w/w), 0.2% (w/w), 0.3% (w/w), 0.4% (w/w), 0.5% (w/w), 0.6% (w/w), 0.7% (w/w), 0.8% (w/w), 0.9% (w/w), or about 1% (w/w). In some examples, the steroid may have a concentration in the pharmaceutical composition of about 0.1% (w/w).

vi. Excipients and Other Ingredients

The pharmaceutical composition may be an aqueous composition that includes sterile water.

In some embodiments, the pharmaceutical composition may further include polyethylene glycol. In some aspects, the polyethylene glycol may have a molar mass of about 400. The polyethylene glycol may have a concentration in the pharmaceutical composition in an amount from about 1% (w/w) to about 4% (w/w). In some embodiments, the polyethylene glycol may have a concentration in the pharmaceutical composition in an amount from about 1% (w/w) to about 1.5% (w/w), about 1% (w/w) to about 2% (w/w), about 1% (w/w) to about 2.5% (w/w), about 1% (w/w) to about 3% (w/w), about 1% (w/w) to about 3.5% (w/w), about 1% (w/w) to about 4% (w/w), about 1.5% (w/w) to about 4% (w/w), about 2% (w/w) to about 4% (w/w), about 2.5% (w/w) to about 4% (w/w), about 3% (w/w) to about 4% (w/w), about 3.5% (w/w) to about 4% (w/w), about 1.5% (w/w) to about 3% (w/w), or about 1.5% (w/w) to about 2.5% (w/w). In preferred embodiments, the polyethylene glycol has a concentration in the pharmaceutical composition of about 2% (w/w).

In some embodiments, the pharmaceutical composition may further include at least one excipient, such as viscosifying agents, solubilizing agents, suspending agents, thickening agents, tonicity adjusting agents, buffers, preservatives, and other excipients known in the art.

In some embodiments, the pharmaceutical composition may further comprise fragrances. Fragrances suitable for use in topical compositions are generally well-known in the art.

B. Package

The packaged composition of the present disclosure includes a package. The package comprises an opaque airless container that is filled with the composition. Without being bound by theory, oxidation and light provide the main pathways of degradation for hypochlorous acid. Thus, an opaque airless container may increase the stability and/or maintain the potency of the pharmaceutical composition.

In some embodiments, the package may include a spray pump, a vessel, and a membrane. In some embodiments, the package may operate by actuation of the spray pump. When the spray pump is actuated, the pharmaceutical composition releases from the spray pump. Air is not introduced into the pump to replace the volume of the sprayed pharmaceutical composition; rather, the membrane within the vessel is forced upward when the spray of the pharmaceutical composition releases from the spray pump. The volume of the vessel is then effectively reduced and no air re-enters the vessel.

In some embodiments, actuating the spray pump may release about 0.1 mL to about 0.2 mL of the pharmaceutical composition. In some aspects, actuating the spray pump may release about 0.1 mL to about 0.12 mL, about 0.12 mL to about 0.14 mL, about 0.14 mL to about 0.16 mL, about 0.16 mL to about 0.18 mL, or about 0.18 mL to about 0.2 mL of the pharmaceutical composition. In some additional aspects, actuating the spray pump may release about 0.1 mL, 0.11 mL, 0.12 mL, 0.13 mL, 0.14 mL, 0.15 mL, 0.16 mL, 0.17 mL, 0.18 mL, 0.19 mL, or about 0.2 mL of the pharmaceutical composition.

In some embodiments, the package may comprise glass, polymers, or other materials suitable for packaging pharmaceutical compositions. In some aspects, the polymers may include polyethylene terephthalate, low density polyethylene, polypropylene, or other polymers known in the art and combinations thereof.

In preferred embodiments, the package meets USP acceptance criteria for light-resistant packaging components. In some aspects, the percentage of spectral transmission through the package at any wavelength of light between 290 nm and 450 nm may be 25% or less, 20% or less, 15% or less, or 10% or less. In some additional examples, the percentage of spectral transmission at any wavelength of light between 290 nm and 450 nm is less than 10%.

C. Stability of the Pharmaceutical Composition

The pharmaceutical composition of the present disclosure packaged in an opaque airless container may be stable for greater than 30 days after the opaque airless spray container is actuated for the first time. In some aspects, the pharmaceutical composition may be stable for at least about 30 days, 60 days, or about 90 days after the opaque airless spray container is actuated for the first time.

In some embodiments, the hypochlorous acid may have a concentration in the pharmaceutical composition of at least about 200 ppm for at least 30 days after the airless spray container is actuated for the first time. In some aspects, the hypochlorous acid may have a concentration in the pharmaceutical composition of at least about 200 ppm for at least about 30 days, at least about 60 days, or at least about 90 days after the pharmaceutical opaque airless spray container is actuated for the first time.

### II. Methods

A. Method of Cleansing and Soothing an Irritated Eyelid

Further described herein are methods for cleansing and soothing an irritated eyelid. The method includes providing a packaged composition of the present disclosure (See Section I above), actuating the opaque airless spray container, and applying the pharmaceutical composition to the irritated eyelid.

In some embodiments, the method may be efficacious in treating a disease or disorder. In some embodiments, the disease or disorder may include dry eye, meibomian gland dysfunction, blepharitis, demodicosis, and/or eczema.

In some embodiments, the step of actuating the opaque airless spray container may release about 0.1 to about 0.2 mL of the pharmaceutical composition. In some aspects, the step of actuating the spray pump may release about 0.1 mL to about 0.12 mL, about 0.12 mL to about 0.14 mL, about 0.14 mL to about 0.16 mL, about 0.16 mL to about 0.18 mL, or about 0.18 mL to about 0.2 mL of the pharmaceutical composition. In some additional aspects, the step of actuating the spray pump may release about 0.1 mL, 0.11 mL, 0.12 mL, 0.13 mL, 0.14 mL, 0.15 mL, 0.16 mL, 0.17 mL, 0.18 mL, 0.19 mL, or about 0.2 mL of the pharmaceutical composition.

In some embodiments, the step of actuating the opaque airless spray container may be performed one or more times before the step of applying the pharmaceutical composition to the irritated eyelid. In some examples, this may be done when large volumes (i.e., larger than 0.1 mL to 0.2 mL) of the pharmaceutical composition are needed to provide a therapeutic or soothing effect.

In some embodiments, the method may further include providing an absorbing article. An absorbing article may be used in lieu of spraying the pharmaceutical composition directly onto the irritated eyelid. In some aspects, the absorbing article may include a cotton ball, a cotton swab, a cotton oval, a cotton pad, a cloth, a handkerchief, a facial tissue, or other absorbing articles known in the art. In some additional aspects, the method may further include actuating the opaque airless spray container in a manner such that the pharmaceutical composition is released onto and absorbed by the absorbing article. In still additional aspects, the method may include applying the absorbing article to the irritated eyelid, wherein the absorbing article has absorbed the pharmaceutical composition.

B. Method of Treatment

Further described herein is a method of treating dry eye, meibomian gland dysfunction, blepharitis, demodicosis, and/or eczema in a patient's eyelid. The method includes providing a packaged composition of the present disclosure (see Section I); actuating the opaque airless spray container, and applying the composition to the patient's eyelid.

In some aspects, the disease or disorder may be caused by a bacteria. The bacteria may include Staphylococcus (e.g., Staphylococcus aureus, MRSA, Staphylococcus epidermis), Streptococcus (e.g., Streptococcus viridans), Micrococcus luteus, Bacillus, Corynebacterium (e.g., Corynebacterium macginleyi), Lysinibacillus, Sphingomonas, Cutibacterium acnes, Micrococcus, Haemophilus, Neisseria, Moraxella, or other bacteria known to cause or complicate dry eye, meibomian gland dysfunction, blepharitis, demodicosis, and/or eczema.

In some aspects, the disease or disorder may be caused by a virus. The virus may include herpes virus (e.g., herpes simplex virus), murine leukemia virus, papillomavirus, SARS-CoV-2, molluscum contagiosum virus, or other viruses known to cause or complicate dry eye, meibomian gland dysfunction, blepharitis, demodicosis, and/or eczema.

In some aspects, the disease or disorder may be caused by a parasite. The parasite may be Demodex, Pthirus pubis, or other parasites known to cause or complicate dry eye, meibomian gland dysfunction, blepharitis, demodicosis, and/or eczema.

In some aspects, the disease or disorder may be caused by a fungus. The fungus may be Malassezia, Candida (e.g., Candida albicans or Candida parapsilosis), Aspergillus (e.g., Aspergillus flavus), Penicillium, Pityrosporum, Rhizopus, or other fungi known to cause or complicate dry eye, meibomian gland dysfunction, blepharitis, demodicosis, and/or eczema.

In some embodiments, the step of actuating the opaque airless spray container may release about 0.1 to about 0.2 mL of the pharmaceutical composition. In some aspects, the step of actuating the spray pump may release about 0.1 mL to about 0.12 mL, about 0.12 mL to about 0.14 mL, about 0.14 mL to about 0.16 mL, about 0.16 mL to about 0.18 mL, or about 0.18 mL to about 0.2 mL of the pharmaceutical composition. In some additional aspects, the step of actuating the spray pump may release about 0.1 mL, 0.11 mL, 0.12 mL, 0.13 mL, 0.14 mL, 0.15 mL, 0.16 mL, 0.17 mL, 0.18 mL, 0.19 mL, or about 0.2 mL of the pharmaceutical composition.

In some embodiments, the step of actuating the opaque airless spray container may be performed one or more times before the step of applying the pharmaceutical composition to the patient's eyelid. In some examples, this may be done when large volumes (i.e., larger than 0.1 mL to 0.2 mL) of the pharmaceutical composition are needed to provide a therapeutic or soothing effect.

In some embodiments, the method may further include providing an absorbing article. An absorbing article may be used in lieu of spraying the pharmaceutical composition directly onto the irritated eyelid. In some aspects, the absorbing article may include a cotton ball, a cotton swab, a cotton oval, a cotton pad, a cloth, a handkerchief, a facial tissue, or other absorbing articles known in the art. In some additional aspects, the method may further include actuating the opaque airless spray container in a manner such that the pharmaceutical composition is released onto and absorbed by the absorbing article. In still additional aspects, the method may include applying the absorbing article to the patient's eyelid, wherein the absorbing article has absorbed the pharmaceutical composition.

C. Method of Removing Makeup

Further described herein is a method of removing makeup. The method includes providing a packaged composition of the present disclosure (see Section I); providing an absorbing article, actuating the opaque airless spray container such that the pharmaceutical composition is released onto the absorbing article, and rubbing the composition on makeup-covered skin.

In some embodiments, the step of actuating the opaque airless spray container may release about 0.1 to about 0.2 mL of the pharmaceutical composition. In some aspects, the step of actuating the spray pump may release about 0.1 mL to about 0.12 mL, about 0.12 mL to about 0.14 mL, about 0.14 mL to about 0.16 mL, about 0.16 mL to about 0.18 mL, or about 0.18 mL to about 0.2 mL of the pharmaceutical composition. In some additional aspects, the step of actuating the spray pump may release about 0.1 mL, 0.11 mL, 0.12 mL, 0.13 mL, 0.14 mL, 0.15 mL, 0.16 mL, 0.17 mL, 0.18 mL, 0.19 mL, or about 0.2 mL of the pharmaceutical composition.

In some embodiments, the step of actuating the opaque airless spray container may be performed one or more times before the step of applying the pharmaceutical composition to the patient's eyelid. In some examples, this may be done when large volumes (i.e., larger than 0.1 mL to 0.2 mL) of the pharmaceutical composition are needed to provide soothing effect or if a large amount of makeup is to be removed.

In some embodiments, the absorbing article may include a cotton ball, a cotton swab, a cotton oval, a cotton pad, a cloth, a handkerchief, a facial tissue, or other absorbing articles known in the art.

The makeup-covered skin of the method includes the eyelid. In some embodiments, the makeup-covered skin may include other areas of the face, including the cheeks, forehead, eyebrows, nose, chin, and temples.

In some embodiments, the makeup may include blush, bronzer, concealer, contour powder or creams, eye shadow, eyelash glue, eyebrow pencil, eyebrow gel, eyebrow powder, eye primer, eyelid primer, face powder, face primer, foundation, foundation alternatives (e.g., bb cream or cc cream), highlight, highlighter, lipgloss, lip-balm, lip liner, lip plumper, lip primers, lip shine, lip stain, lip sticks, mascara, rouge, setting powder, setting spray, or other makeup known to those having skill in the art.

### EXAMPLES

### Example 1

A pharmaceutical composition of the present disclosure was made using the following procedure. First, an L-menthol stock solution was made by dissolving 10% (w/w) L-menthol in polyethylene glycol-400. The solution was mixed until completely dissolved.

Next, a 1 L mixing vessel was filled to 90% final volume with a 0.05% hypochlorous acid solution. Sodium lauryl sarcosinate was added to the hypochlorous acid solution and mixed until homogenous. Then, the L-menthol stock solution was added and mixed until homogeneous.

The pH of the solution was measured. The pH was then adjusted as needed to between 4.5 and 5.5 by adding hydrochloric acid.

Next, sterile water was added to the solution to bring the solution to the final volume, yielding a final hypochlorous acid concentration of about 0.02% (w/w), a final menthol concentration of about 0.03% (w/w), and a final sodium lauryl sarcosinate concentration of about 1% (w/w).

Finally, the solution was packaged in an opaque airless spray container. The pump of the container was primed to remove all remaining air from the container.

What is claimed is:

1. A packaged eyelid or eyelash scrub composition, the packaged composition comprising:
a pharmaceutical composition comprising hypochlorous acid and menthol; and
a package comprising an opaque airless spray container filled with the pharmaceutical composition,
such that the pharmaceutical composition is stable for greater than 30 days after the airless spray container is actuated for the first time.

2. The packaged composition of claim 1, wherein the pharmaceutical composition has a viscosity from about 1 cP to about 10 cP.

3. The packaged eyelid scrub composition of claim 1, wherein the menthol is L-menthol.

4. The packaged eyelid scrub composition of claim 3, wherein the L-menthol has a concentration in the pharmaceutical composition from about 0.01% (w/w) to about 0.03% (w/w).

5. The packaged eyelid scrub composition of claim 1, wherein the hypochlorous acid has a concentration in the pharmaceutical composition from about 0.01% (w/w) to about 0.05% (w/w).

6. The packaged eyelid scrub composition of claim 1, wherein the pharmaceutical composition further comprises at least one surfactant.

7. The packaged eyelid scrub composition of claim 7, wherein the at least one surfactant is a non-ionic surfactant, an anionic surfactant, or combinations thereof.

8. The packaged eyelid scrub composition of claim 8, wherein the anionic surfactant is sodium lauryl sarcosinate.

9. The packaged eyelid scrub composition of claim 8, wherein the non-ionic surfactant is sorbitan monolaurate.

10. The packaged eyelid scrub composition of claim 7, wherein the at least one surfactant forms micelles.

11. The packaged eyelid scrub composition of claim 7, wherein the at least one surfactant has a concentration in the pharmaceutical composition of between about 0.1% (w/w) to about 2% (w/w).

12. The packaged eyelid scrub composition of claim 1, wherein the pharmaceutical composition further comprises a pH modifier.

13. The packaged eyelid scrub composition of claim 12, wherein the pH modifier is selected from the group consisting of hydrochloric acid and sodium hydroxide.

14. The packaged eyelid scrub composition of claim 1, wherein the pharmaceutical composition has a pH from about 4 and about 7.

15. The packaged eyelid scrub composition of claim 1, wherein the opaque airless container comprises glass, polyethylene terephthalate, low density polyethylene, polypropylene, or combinations thereof.

16. The packaged eyelid scrub composition of claim 1, wherein the opaque airless container meets USP acceptance criteria for light-resistant packaging components.

17. The packaged eyelid scrub composition of claim 1, wherein the percentage of spectral transmission of the opaque airless spray container the opaque airless spray container at any wavelength of light between 290 nm and 450 nm is 25% or less.

18. The packaged eyelid scrub composition of claim 17, wherein the percentage of spectral transmission of the opaque airless spray container at any wavelength of light between 290 nm and 450 nm is 10% or less.

19. The packaged eyelid scrub composition of claim 1, wherein the hypochlorous acid has a concentration in the pharmaceutical composition of at least about 200 ppm for at least 30 days after the opaque airless spray container is actuated for the first time.

20. The packaged eyelid scrub composition of claim 1, wherein the pharmaceutical composition further comprises an active ingredient.
